(19) United States
(12) Patent Application Publication
(10) Pub. No.: US 2007/0141121 A1
(43) Pub. Date: Jun. 21, 2007

(54) Method of treating kidney disease

(76) Inventor: Gary Jim Calton, Elkridge, MD (US)

Correspondence Address: Gary J. Calton, 5331 Landing Road, Elkridge, MD, 21075, US

(21) Appl. No.: 11/312,326

(22) Filed: Dec. 20, 2005

(51) Int. Cl. A61K 47/00

(52) U.S. Cl. 424/439

(57) ABSTRACT

The progression of kidney disease and especially polycystic kidney disease in humans may be retarded and the symptoms ameliorated by administration of an effective amount of soy molasses or isolated soyasaponins or mixtures thereof in conjunction with the essential amino acids. Restriction of protein to a moderate level of 0.8 g/kg/day or less is a preferable adjunct to this treatment.

### FEDERALLY SPONSORED RESEARCH

Not Applicable

### SEQUENCE LISTING OR PROGRAM

Not applicable

### BACKGROUND OF THE INVENTION—FIELD OF THE INVENTION

The present invention relates to methods and compositions for treating kidney disease, especially polycystic kidney disease.

### BACKGROUND OF THE INVENTION—PRIOR ART

Autosomal Polycystic Kidney Disease (ADPKD) is the most prevalent, potentially fatal, inherited disease affecting the human kidney with a reported incidence of 1 out of every 500 births. It has been estimated that 500,000 persons in North America suffer from this disease. Worldwide, almost 5 million persons suffer from this disease. Polycystic kidney disease (PKD) results in enlarged, cyst-filled kidneys in the abdomen producing severe back pain, early and progressive hypertension, frequent urinary tract infections and blood in the urine. PKD also has inflammatory and other clinical components. Approximately 45% of cases can be expected to progress to end-stage renal disease (fatal) before the age of 60, adding to health care costs for dialysis treatment, hospitalization, premature morbidity due to cardiovascular and other disorders, surgeries, etc.

At present, there are no known treatments of PKD other than renal ablation, organ transplantation and cyst decompression, all costly, and sometimes life threatening. There is some evidence that, in adult humans, a slower rate of kidney deterioration is associated with a moderate protein restriction (Oldrizzi, L., et al., 1985., Kidney Int., 27(3):553-7). Holub (U.S. Pat. No. 6,784,159) states that this protein restriction is of limited use in the growing child with PKD due to the need for protein in growth.

Philbrick, D J et al. (2003, Kidney International, 63:1230), have shown that feeding a soyasapoginin isolated from soy molasses by alcohol extraction retarded disease progression in a murine model of polycystic kidney disease as well as feeding soy protein isolate markedly slowed disease progression in the DBA/2FG-pcy(pcy) mouse model of ADPKD, however they combined this soyasapoginin with casein as their source of protein.

Sakemi et al. (2001, American Journal of Kidney Disease, 37:832) showed a that semipurified alcohol extract of soy protein in a casein diet gave greater retention of renal function as indicated by lower blood urea nitrogen and greater creatinine clearance less glomerular hypertrophy and less renal histological damage in comparison to a casein diet.

Fair et al., suggested that studies with purified amino acid diets to aid in determining whether the different protein effect is due to a beneficial effect of soy protein or a detrimental effect of casein (Fair D E, Ogborn M R, Weiler H A, Bankovic-Calic N, Nitschmann E P, Fitzpatrick-Wong S C, Aukema H M. Dietary soy protein attenuates renal disease progression after 1 and 3 weeks in Han:SPRD-cy weanling rats. J Nutr. 2004 June; 134(6):1504-7.). Although these investigators were extremely skilled in the art and were aware of the effect of soyasaponins reported by Philbrick et al., they did not suggest the use of essential amino acids in conjunction with alcoholic soyasaponin extracts of soy molasses or of isolated components thereof to prevent the deleterious effects of protein.

It has previously been shown that patients suffering from kidney disease who practice a very low protein diet supplemented by either essential amino acids or keto analogues thereof, including those patients having polycystic kidney disease, undergo amelioration of the disease. (Walser M. Is there a role for protein restriction in the treatment of chronic renal failure? Blood Purif. 2000;18(4):304-12). However, there is also considerable disbelief in the expert community of nephrologists about the efficacy of this treatment (Bircher G. The conservative management of chronic renal failure: results of a recent survey. J Ren Nutr. 1998 April;8(2):83-7; Mehrotra R, Nolph K D. Treatment of advanced renal failure: low-protein diets or timely initiation of dialysis? Kidney Int. 2000 October;58(4):1381-8).

### DETAILED DESCRIPTION OF THE INVENTION

Soyasaponin extracts and the individual components thereof, are known to be useful in treating kidney disease (Holub, U.S. Pat. No. 6,784,159 and Shinohara et al U.S. Pat. No. 4,217,345, incorporated herein by reference, ). Holub supplemented a casein-based diet with a crude saponin-enriched alcohol extract (SEAE) from a soy protein isolate and achieved a 25% reduction in total kidney weight, a marker for disease progression in several rodent models of polycystic kidney disease (PKD). Kidney morphometric analyses showed that mean cyst volume, and hence cyst size, was significantly reduced in the kidneys from the SEAE-fed group. Plasma creatinine and urea contents were lowered by the SEAE-fed group, which suggests that renal function in these animals had not declined to the same extent as that observed in casein-fed mice (Philbrick et al.) and ascribed these beneficial effects to the saponins derived from soybeans. The investigators in these studies were all experts in nutrition and kidney disease, however, it was not obvious to these investigators who were exceptionally skilled in their art that protein restriction or supplementation with amino acids in conjunction with the soyasaponin extract would be of value and instead used a high protein supplement (casein) in all trials.

Modified protein diets are thought to slow progression of kidney disease. However, their use is especially controversial in kidney patients (Lim V S, Flanigan M J. Protein intake in patients with renal failure: comments on the current NKF-DOQI guidelines for nutrition in chronic renal failure. Seminars in Dialysis 2001 May-June;14(3):150-2). Modified protein diets are said to be contraindicated in pediatric patients (Sedman A, Friedman A, Boineau F, Strife C F, Fine R. Nutritional management of the child with mild to moderate chronic renal failure. J Pediatr. 1996 August;129(2):s13-8.).

Thus, it has not been envisioned by those skilled in the art that the combination of an essential amino acid supplement which is capable of replacing protein nutrition (Taylor T P, Morris J G, Willits N H, Rogers Q R. Optimizing the pattern of essential amino acids as the sole source of dietary nitrogen supports near-maximal growth in kittens. J Nutr. 1996 September;126(9):2243-52) used in conjunction with a soyasaponin extract, selected saponins mixtures or individual soyasaponins isolated therefrom would improve the treatment of kidney patients and especially that of pediatric kidney patients and more especially polycystic kidney patients.

The component essential amino acids used in this invention are especially useful in the weight ratio of L-histidine 45 mg, L-isoleucine 60 mg, L-leucine 90 mg, L-lysine 65 mg, L-methionine 90 mg, L-phenylalanine 70 mg, L-threonine 65 mg, L-tryptophan 25 mg, L-tyrosine 75 mg, and L-valine 135 mg. The corresponding keto analogs of these amino acids may also be substituted for any or all of the amino acids in the same relative ratio.

It will be appreciated that the invention contemplates variations in the amounts of amino acids or keto acids from those recited above. Typically the variation can be in the order of 10-30% by weight. However, it is preferred that the indicated ratios of the amino acids or keto acids be maintained. Thus, for example, the amounts of L-leucine and L-methionine should be about twice the amount of L-histidine administered while the amount of L-isoleucine should be about two-thirds of the L-leucine and L-methionine.

An essential amino supplement replacing some to most of the protein in the diet, supplied by either tablet or other oral supplementation method, in conjunction with the administration of an soyasaponin extract or multiple or individual components of said extract, will lessen the deleterious effects of the soy or other protein in the diet of kidney patients and especially those with polycystic kidney disease, in addition to providing the desirable reduction in cyst growth and kidney size. This is especially useful in the pediatric polycystic kidney patient where a sufficient protein level is required to achieve growth.

Accordingly, the present invention provides a method of treating a kidney disease comprising administering an effective amount of soy extract such as that provided in U.S. Pat. Nos. 4,217,345, 4,557,927, 6,355,816, 5,919,921 or 6,784,159, incorporated herein by reference, or individual components derived therefrom as described in U.S. Pat. Nos. 4,557,927, 6,784,159, incorporated herein by reference, or an analog thereof in conjunction with a keto analog of the essential amino acids as described in U.S. Pat. Nos. 4,100,161, 4,320,146, 4,352,814, incorporated herein by reference, or an essential amino acid supplement as described in U.S. Patent Applications 20020187180, 20020193342, 20030185876, 20040197401, and U.S. Pat. No. 6,713,501, incorporated herein by reference, to an animal in need thereof. The invention also includes the use of a low protein or low phosphorous diet in conjunction with the aforementioned soy components and amino acid or keto acid supplement to treat kidney disease and especially polycystic kidney disease.

### SUMMARY OF THE INVENTION

An object of this invention is to provide a method for treating kidney disease whose active components are an essential amino acid supplement in conjunction with a soyasaponin extract or multiple or individual components thereof.

Another object of this invention is to provide a method for treating kidney disease whose active components are a supplement of the keto analogs of the essential amino acids in conjunction with a soyasaponin extract or multiple or individual components thereof.

Yet another object of this invention is to provide a method for treating polycystic kidney whose active components are an essential amino acid supplement in conjunction with a soyasaponin extract or multiple or individual components thereof.

A further object of this invention is to provide a method for treating polycystic kidney whose active components are a supplement of the keto analogs of the essential amino acids in conjunction with a soyasaponin extract or multiple or individual components thereof.

This invention also provides for the addition of a protein-restricted diet which is desirable for reducing symptoms in patients severely affected with chronic renal failure. In such cases, the level of dietary protein is preferably restricted to less than 0.8 grams of protein per day per kilogram of ideal body weight and more preferably to less than 0.6 grams of protein per day per kilogram of ideal body weight.

Accordingly, the present invention provides a method of treating a kidney disease and more especially polycystic kidney disease comprising administering an effective amount of a soyasaponin extract or multiple components thereof in conjunction with a balanced supplement of the essential amino acids or their keto analogs to an animal in need thereof.

A preferred variation of the aforementioned amino acid or keto analog supplement and soyasaponin extract or multiple components thereof is their use in conjunction with a modified protein diet in which the amount of protein in the diet is less than 0.8 g/kg of body weight.

The amino acid supplement of the invention is a mixture of essential amino acids comprising L-histidine 45 mg, L-isoleucine 60 mg, L-leucine 90 mg, L-lysine 65 mg, L-methionine 90 mg, L-phenylalanine 70 mg, L-threonine 65 mg, L-tryptophan 25 mg, L-tyrosine 75 mg, and L-valine 135 mg, or their keto analogs or mixtures thereof, administered in a dose, either singly or divided, of 0.05 to 0.3 grams per kilogram of body weight per day.

To illustrate the invention, a group of polycystic kidney disease patients is randomized to receive either the essential amino acids or their keto analogs in conjunction with an effective dosage of soyasaponin extract or multiple or individual components or a similar placebo for three months with no change in their diet. Before supplementation with the essential amino acids or their keto analogs or a mixture thereof in conjunction with the administration of a soyasaponin extract or multiple or individual components thereof and at monthly intervals during supplementation, serum albumin, serum creatinine, BUN, back pain, anthropometry, and serum amino acid levels are measured. Patients receiving the amino acid or keto acid or mixture thereof supplement in conjunction with the soyasaponin extract or multiple components thereof should exhibit fewer symptoms of polycystic kidney disease than patients receiving the placebo.

Other features and advantages of the present invention will become apparent from the following detailed description. It should be understood, however, that the detailed description and the specific examples while indicating preferred embodiments of the invention, are given by way of illustration only, since various changes and modifications within the spirit and scope of the invention will become apparent to those skilled in the art from this detailed description.

I claim:

1. A method for retarding the progression of polycystic kidney disease comprising the administration of an agent selected from the group of agents consisting of a soyasaponin fraction, or an individual component thereof with a supplement selected from the group of amino acids consisting of valine, phenylalanine, methionine, leucine, isoleucine, tryptophan, lysine, histidine, tyrosine and threonine, or the keto analogs of said essential amino acids, said keto analogs being present in the form of the alpha-keto acids per se or as salts of the alpha-keto acids.

2. The method of claim 1 additionally comprising a modified protein diet wherein said diet is restricted to less than 0.8 g protein per kilogram of body weight per day to said patients.

3. The method according to claim 2, wherein said modified protein diet is restricted to less than about 0.6 grams of protein daily per kilogram of body weight.

4. A method according to claim 1, wherein said agent is administered while said human is on a phosphorus-restricted diet.
